(19) United States
(12) Patent Application Publication
(10) Pub. No.: US 2025/0032748 A1
(43) Pub. Date: Jan. 30, 2025

(54) TRANS-RADIAL ACCESS ENDOVASCULAR CATHETERS AND METHODS OF USE

(71) Applicant: Daniel Ezra Walzman, Teaneck, NJ (US)

(72) Inventor: Daniel Ezra Walzman, Teaneck, NJ (US)

(21) Appl. No.: 18/912,791

(22) Filed: Oct. 11, 2024

Related U.S. Application Data
(63) Continuation of application No. 17/423,502, filed on Jul. 16, 2021.
Application No. 17/423,502 is a continuation of application No. 16/602,469, filed on Oct. 11, 2019.
Application No. 16/602,469 is a continuation of application No. 16/600,096, filed on Oct. 11, 2019, now abandoned.

(51) Int. Cl. A61M 25/00; A61B 5/00; A61B 5/055; A61B 5/283; A61B 6/00; A61B 6/50; A61F 2/856; A61F 2/95; A61F 2/954; A61M 25/04; A61M 25/10

(52) U.S. Cl. CPC A61M 25/0041; A61B 5/4836; A61B 5/6852; A61F 2/856; A61F 2/954; A61M 25/0052; A61M 25/10; A61B 5/055; A61B 5/283; A61B 6/481; A61B 6/487; A61B 6/504; A61F 2002/9528; A61M 25/04; A61M 2205/0266; A61M 2210/12

(57) ABSTRACT

Devices and methods for establishing trans-radial access for medical intervention are described. In particular, some embodiments are optimized for consistently safely achieving complete cerebral angiography via a single trans-radial access site. A system may include a catheter with at least two active steering sites. Some embodiments may include at least one balloon. The methods include using said steering mechanisms to help guide and support said catheter. In some embodiments, additional use is made of at least one vascular arch to provide further support and prevent kickback and prolapse of said catheter and any additional devices passed therethrough.

### FIELD OF THE INVENTION

The present disclosure relates generally to endovascular devices and methods of use during a medical procedure. More particularly, the present disclosure describes endovascular devices and methods for establishing expanded trans-radial vascular access as alternatives to traditional catheterization.

### BACKGROUND OF THE INVENTION

Access to patient blood vessels is necessary for a wide variety of medical, diagnostic, and/or therapeutic purposes. While a wide variety of variations exist, the basic technique relies on access via a long and tortuous path. Craniofacial angiography and cardiac catheterization, for example, are often performed through a transfemoral route. More recently, however, a trans-radial approach has been developed for cardiac catheterization. Studies have demonstrated that trans-radial vascular access has lower access-site major complication rates than transfemoral access. However, current catheter technology can complicate trans-radial access to contralateral carotid, vertebral circulations, and ipsilateral carotid circulations in certain patients.

With the introduction of a greater number and variety of intravascular techniques, including angioplasty, atherectomy, endovascular aneurysm repair, thrombectomy, minimally invasive cardiac surgery, and the like, a need has arisen to for additional access ports for various procedures. The devices and methods described herein improve upon traditional catheterization so as to reduce the complexities and costs of such procedures.

The prior art discloses the use of trans-radial arterial catheterization. Trans-radial for medical purposes means through, by way of, or employing the radial artery. More specifically, trans-radial access is used to perform medical catheterization procedures and for therapeutic procedures.

More recently, trans-radial access for medical intervention has become increasingly popular given the significant reduction in trauma and blood loss, even with aggressive use of anticoagulation and antiplatelet therapies. Often during such procedures, patients are given high doses of blood thinners and platelet inhibiting medications.

While the present disclosure is described in the context of trans-radial cerebral angiography, it should be understood that the devices, methods, and principles described herein may be utilized in a variety of applications and medical procedures, such as, for example, the catheterization of a contralateral internal mammary artery, which is often an important vessel needed to be accessed to do a complete heart catheterization in patients with prior bypass surgery. The devices described herein, however, can also be used via other access sites as well.

The devices can used as sheath to access all “great vessels” of the aortic arch via a unilateral radial artery approach, while also providing an inner lumen through which additional wires and catheters can be advanced into all “selective cerebral angiography vessels (bilateral internal carotid artery, bilateral vertebral artery, bilateral external carotid artery) and even beyond—into the brain and head and neck for interventions.

Radial artery access is known in the art and is typically achieved with a short, bevel 21-gauge needle, and typically, a 0.018-0.021 guide wire. This smaller needle system allows for better control and pulsatile blood flow can be seen immediately. It is suggested during a radial artery catheterization to use a smaller needle than one traditionally used during femoral catheterization, which may reduce difficulty when obtaining access.

There are a variety of sheaths available on the market that may be suitable for radial access. There are some characteristics, however, that may be desired in a radial sheath such as a tapered edge and hydrophilic coating. The tapered edge allows for smooth insertion of the sheath, and a hydrophilic coating on the sheath reduces the incidence of radial artery spasm during trans-radial coronary procedures.

Although a JL 4 and JR 4 catheter can be used for left and right coronary artery cannulation, there are catheters on the market by various vendors designed specifically for radial artery access. These catheters have the common characteristic of a primary and secondary curve. A radial-specific catheter enables angiography of both right and left coronaries with a clockwise and counterclockwise rotation of one catheter. Eliminating catheter exchange can result in less total procedure time as well as fluoroscopy time and less incidence of radial artery spasm.

Additionally, the prior art discloses a set of Walzman radial access catheters (e.g., U.S. patent application Ser. No. 16/501,591), which facilitate percutaneous access to either carotid artery safely in the vast majority of patients and a reduction in access-site complications.

### SUMMARY OF THE INVENTION

In one aspect of the present disclosure, improved devices and methods are described for establishing trans-radial (e.g., arterial) access to a multitude of end vessels, via a single site of radial access. While access can be established to a variety of particular blood vessels, including both arteries and veins, such as the femoral artery, radial artery, and the like, accessing the radial artery may reduce the overall complexity of the procedure.

The methods and devices described herein offer certain improvements over the techniques and devices disclosed in the prior art. For example, the methods and devices described reduce patient trauma and increase the number of procedures that can be performed via a single radial access site. Notwithstanding the particular advantages of trans-radial access, the devices and methods described herein may also be utilized in connection with alternate access sites, including (but not limited to) the brachial artery, axillary artery, femoral vessels, etc.

In another aspect of the present disclosure, a medical device is disclosed for establishing trans-radial access to a target site in a blood vessel that includes a body and a plurality of pull wires. The body includes: a proximal end hole; a distal end hole that is positioned opposite to the proximal end hole; a working lumen that extends through the body from the proximal end hole to the distal end hole; and a plurality of active segments that are deflectable to reconfigure the medical device between a first configuration, in which the body is generally linear, and a second configuration, in which the body is non-linear. The plurality of pull wires correspond in number to the plurality of active segments such that each active segment is connected to a single pull wire, whereby applying an axial force to each pull wire causes deflection of a corresponding active segment.

In certain embodiments, the plurality of active segments may be spaced axially along a longitudinal axis of the body.

In certain embodiments, the body may further include a plurality of inactive segments.

In certain embodiments, the body may be configured such that the plurality of active segments and the plurality of inactive segments are arranged in a staggered pattern along the longitudinal axis.

In certain embodiments, the body may include: a first inactive segment; a first active segment that is located distally of the first inactive segment; a second inactive segment that is located distally of the first active segment; and a second active segment that is located distally of the second inactive segment.

In certain embodiments, the plurality of pull wires may include a first pull wire that is connected to the first active segment and a second pull wire that is connected to the second active segment.

In certain embodiments, the first active segment and the second active segment may each be configured to define a bend that lies substantially within a range of approximately 0 degrees to approximately 270 degrees. More specifically, in certain embodiments, the first active segment may be configured to define a first bend that lies substantially within a range of approximately 0 degrees to approximately 270 degrees and the second active segment may be configured to define a second bend that lies substantially within a range of approximately 0 degrees to approximately 180 degrees.

In certain embodiments, the first pull wire may be connected to the first active segment such, upon defection, that the first active segment defines a first bend that curves in a first direction.

In certain embodiments, the second pull wire may be connected to the second active segment such, upon deflection, the second active segment defines a second bend that curves in a second direction.

In certain embodiments, the second direction may be generally opposite to the first direction.

In certain embodiments, the medical device may further include an inflatable balloon that is supported on the body proximally of the distal end hole.

In certain embodiments, the body may include an outer wall and a plurality of channels that extend through the outer wall in generally parallel relation to the working lumen.

In certain embodiments, the plurality of channels may correspond in number to the plurality of pull wires such that each channel receives a single pull wire.

In certain embodiments, the medical device may be configured for connection to an inflation source.

In certain embodiments, at least one of the plurality of channels may be configured for communication with the inflation source such that inflation fluid is communicable from the inflation source to the inflatable balloon therethrough.

In another aspect of the present disclosure, a medical device is disclosed for establishing trans-radial access to a target site in a blood vessel that includes a body defining a longitudinal axis and a plurality of pull wires. The body includes a plurality of active segments and a plurality of inactive segments. The plurality of active segments are deflectable to reconfigure the medical device between a generally linear configuration and a non-linear configuration. The body is configured such that the plurality of active segments and the plurality of inactive segments are arranged in a staggered pattern along the longitudinal axis. The plurality of pull wires are connected to the plurality of active segments such that each active segment is deflectable via an axial force applied to a corresponding pull wire. Each active segment is configured to define a bend greater than approximately 90 degrees upon deflection.

In certain embodiments, the plurality of pull wires may include first and second pull wires that are connected to one of the plurality of active segments at respective first and second connection points.

In certain embodiments, the first and second connection points may be located generally opposite to each other to facilitate deflection of the body in generally opposing first and second directions.

In certain embodiments, each active segment may be configured to define a bend between approximately 90 degrees and approximately 270 degrees.

In certain embodiments, the body may include: a first inactive segment; a first active segment that is located distally of the first inactive segment; a second inactive segment that is located distally of the first active segment; and a second active segment that is located distally of the second inactive segment.

In certain embodiments, the first active segment may be configured to define a first bend that lies substantially within a range of approximately 90 degrees to approximately 270 degrees and the second active segment may be configured to define a second bend that lies substantially within a range of approximately 90 degrees to approximately 180 degrees.

The various catheters described herein can include at least one additional lumen that is located in the outer wall of the catheter and that can exit the outer wall of the catheter via at least one perforation to provide irrigation proximal to an inflatable balloon (e.g., when the balloon is inflated), so as to reduce (or entirely eliminate) clot formation and the stasis of blood proximal to the balloon that may otherwise form as a result of vessel occlusion via the balloon.

The devices described herein can be used alone or in combination with additional catheters that may be inserted therethrough into a patient's vasculature to access more distal locations. The presently disclosed devices may also be used to obtain access to a particular target vessel, and may then be exchanged utilizing standard exchange techniques, for a secondary catheter that may be devoid of the (embedded) wires described herein. These secondary catheters may include thinner walls, allowing for a greater inner diameter for a given outer diameter and, thus, allowing more procedures to be performed through a given size access artery.

### BRIEF DESCRIPTION OF THE DRAWINGS

FIG. 1A is a schematic representation of one embodiment of a medical device according to the principles of the present disclosure shown in a first (initial, normal) configuration.

FIG. 1B is a transverse cross-sectional view of the medical device seen in FIG. 1A taken along line 1B-1B.

FIG. 1C is a schematic representation of the medical device of FIG. 1A shown in a second (subsequent, deflected) configuration.

FIG. 1D is a transverse cross-sectional view of an alternate embodiment of the medical device.

FIG. 1E is a schematic representation of an alternate embodiment of the medical device shown in the second (subsequent, deflected) configuration.

FIG. 1F is a transverse cross-sectional view of the embodiment of the medical device seen in FIG. 1E taken along line 1F-1F.

FIG. 1G is a schematic representation of an alternate embodiment of the medical device shown in the first (initial, normal) configuration.

FIG. 1H is a schematic representation of an alternate embodiment of the medical device shown in the first (initial, normal) configuration.

FIG. 1I is a transverse cross-sectional view of the embodiment of the medical device seen in FIG. 1G taken along line 1I-1I.

FIG. 1J is a schematic representation of an alternate embodiment of the medical device shown in the first (initial, normal) configuration.

FIG. 1K is a transverse cross-sectional view of the embodiment of the medical device seen in FIG. 1J taken along line 1K-1K.

FIG. 1L is a schematic representation of an alternate embodiment of the medical device shown in the second (subsequent, deflected) configuration.

FIG. 1M illustrates the vessels proximal to the aorta.

FIG. 2 illustrates a catheter of the present invention using the right radial approach to the right vertebral artery.

FIG. 3 illustrates a catheter of the present invention using the right radial approach to the right internal mammary artery.

FIG. 4 illustrates a catheter of the present invention using the right radial approach to the right carotid artery.

FIG. 5 illustrates a catheter of the present invention using the right radial approach to the left common carotid artery.

FIG. 6 illustrates a catheter of the present invention using the right radial approach to left subclavian artery using an alternate technique.

FIG. 7 illustrates a catheter of the present invention using the right radial approach to the left common carotid artery while implementing an arch fulcrum support configuration.

FIG. 8 illustrates a catheter of the present invention using the right radial approach to the left vertebral artery, employing the lesser curve of the arch of the aorta as a vascular fulcrum.

FIG. 9 illustrates a catheter of the present invention using the right radial approach to the left internal mammary artery.

FIG. 10 illustrates a catheter of the present invention using the right radial approach to the left carotid artery, using an alternate technique, employing the lesser curve of the arch of the aorta as a vascular fulcrum.

FIG. 11 illustrates the same device as FIG. 2 with the additional of a partially inflated traditional balloon or a partially inflated hydrogel balloon.

FIG. 12 illustrates the same device as FIG. 3 with the additional of a partially inflated traditional balloon or a partially inflated hydrogel balloon.

FIG. 13 illustrates the same device as FIG. 4 with the additional of a partially inflated traditional balloon or a partially inflated hydrogel balloon.

FIG. 14 illustrates the same device as FIG. 5 with the additional of a partially inflated traditional balloon or a partially inflated hydrogel balloon.

FIG. 15 illustrates the same device as FIG. 6 with the additional of a partially inflated traditional balloon or a partially inflated hydrogel balloon.

FIG. 16 illustrates the same device as FIG. 7 with the additional of a partially inflated traditional balloon or a partially inflated hydrogel balloon.

FIG. 17 illustrates the same device as FIG. 8 with the additional of a partially inflated traditional balloon or a partially inflated hydrogel balloon.

FIG. 18 illustrates the same device as FIG. 9 with the additional of a partially inflated traditional balloon or a partially inflated hydrogel balloon.

FIG. 19 illustrates the same device as FIG. 10 with the additional of a partially inflated traditional balloon or a partially inflated hydrogel balloon.

### DETAILED DESCRIPTION OF THE INVENTION

The principles of the present disclosure find wide applicability to a variety of medical procedures including, for example, 4 and 6 vessel cerebral angiograms via a single radial artery approach and complete heart catherization via a single radial approach in patients with internal mammary artery bypass on opposite side from radial access (e.g., right radial access and left mammary artery or bilateral mammary artery bypass).

During methods according to the principles of the present disclosure, a catheter is introduced to a patient via an access needle (not shown) that is inserted into a patient's radial artery utilizing a percutaneous trans-radial approach in which the access needle penetrates the skin and is then advanced into the radial artery. Following access to the radial artery via the access needle, an insertion (rail) wire is fed through the access needle and into and through the patient's vessels such that the insertion wire functions as a delivery rail. Once the rail wire is positioned as necessary or desired in a target vessel, the access needle can be removed, leaving the rail wire in place. A primary catheter (sheath) is then advanced over the rail wire and a secondary (working) catheter is then inserted into the primary catheter and fed to the target area such that the secondary catheter provides an access lumen for working wires, balloons, stents, and other medical devices. It is envisioned that the primary catheter may be eliminated and that procedures may be performed vial sole use of the secondary catheter (in an orientation sometimes referred to as “bareback”), which allows a larger working catheter to be used via a trans-radial approach. It is envisioned the devices described herein may configured for use in any configuration.

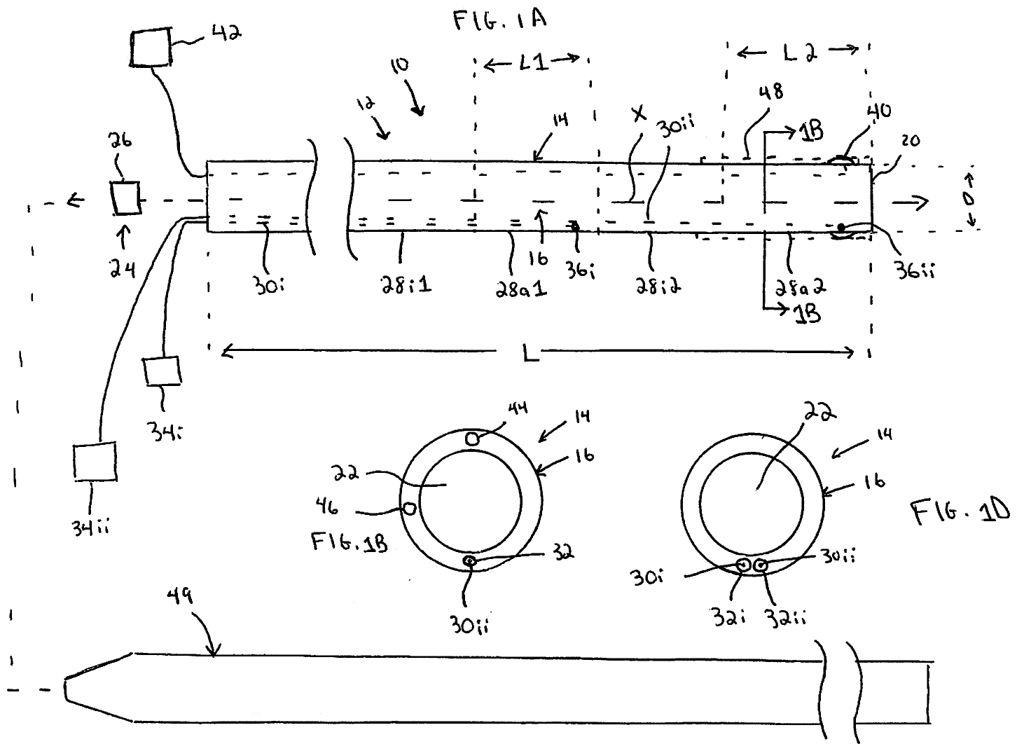

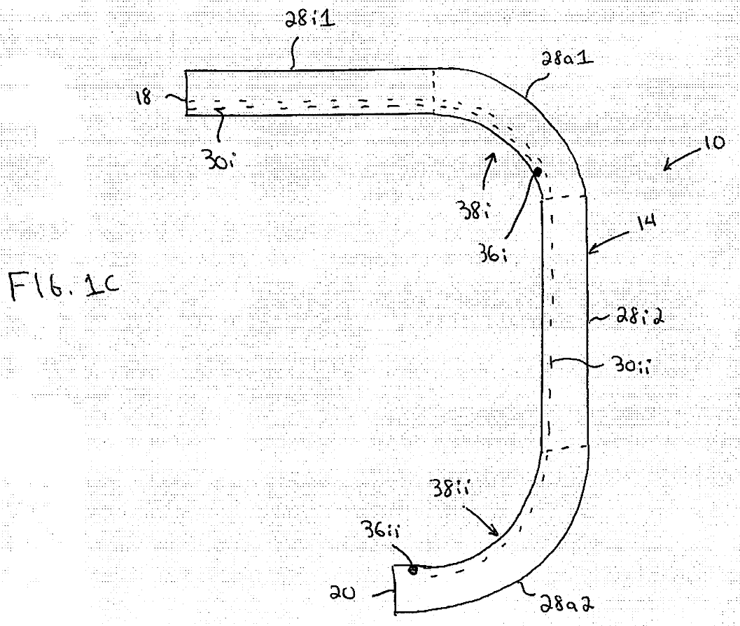

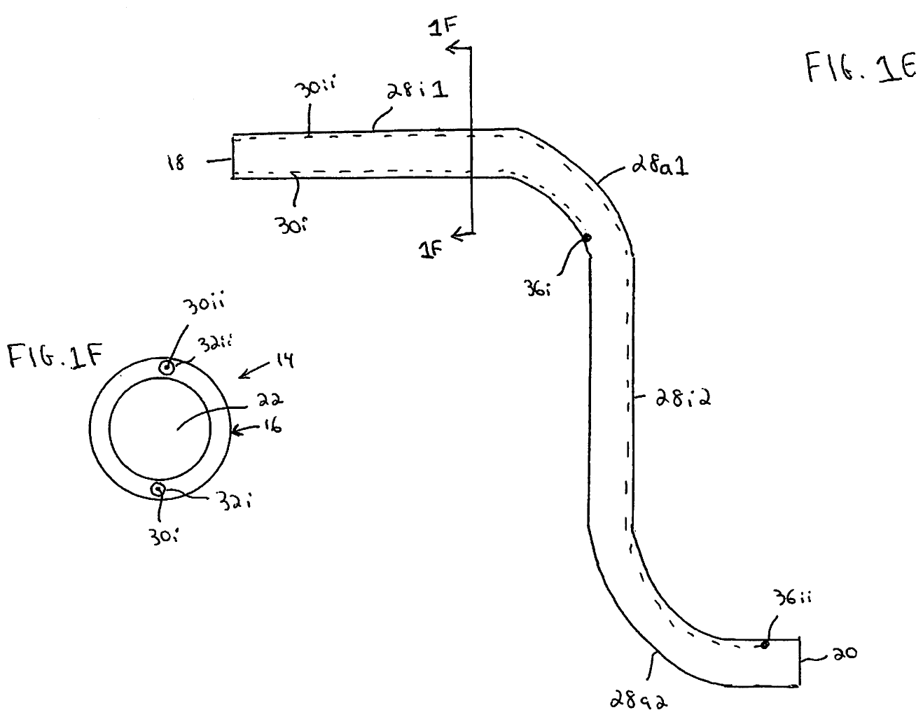

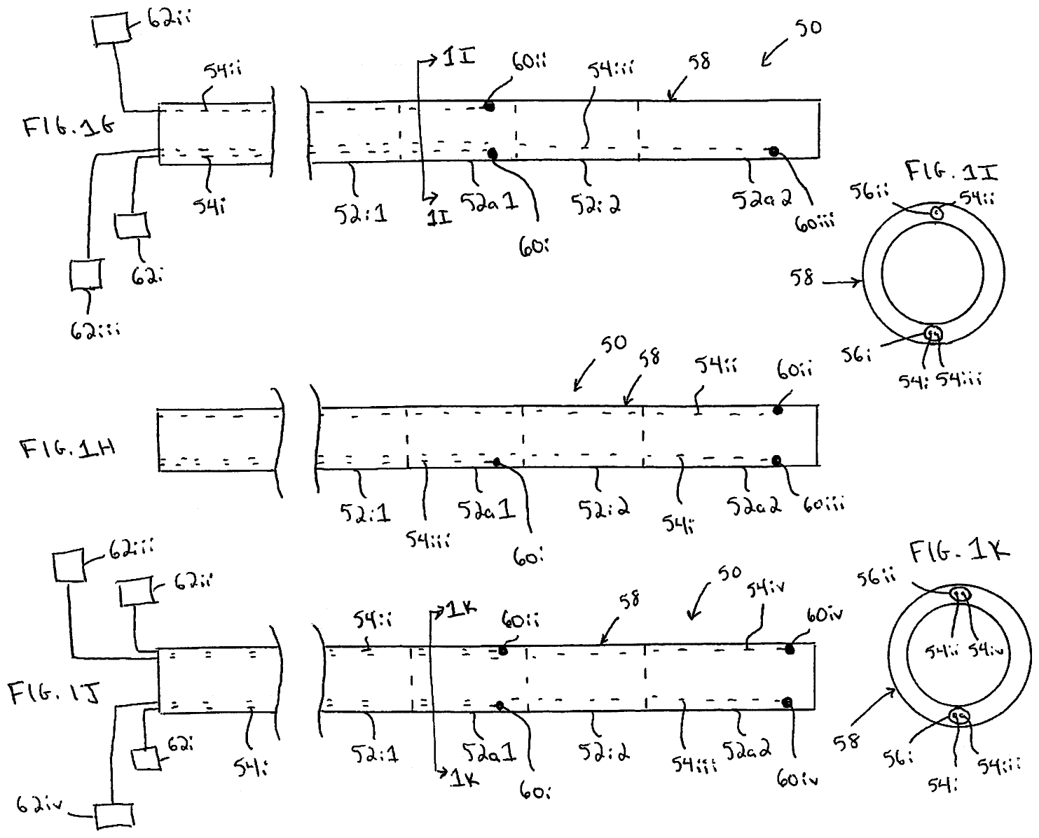

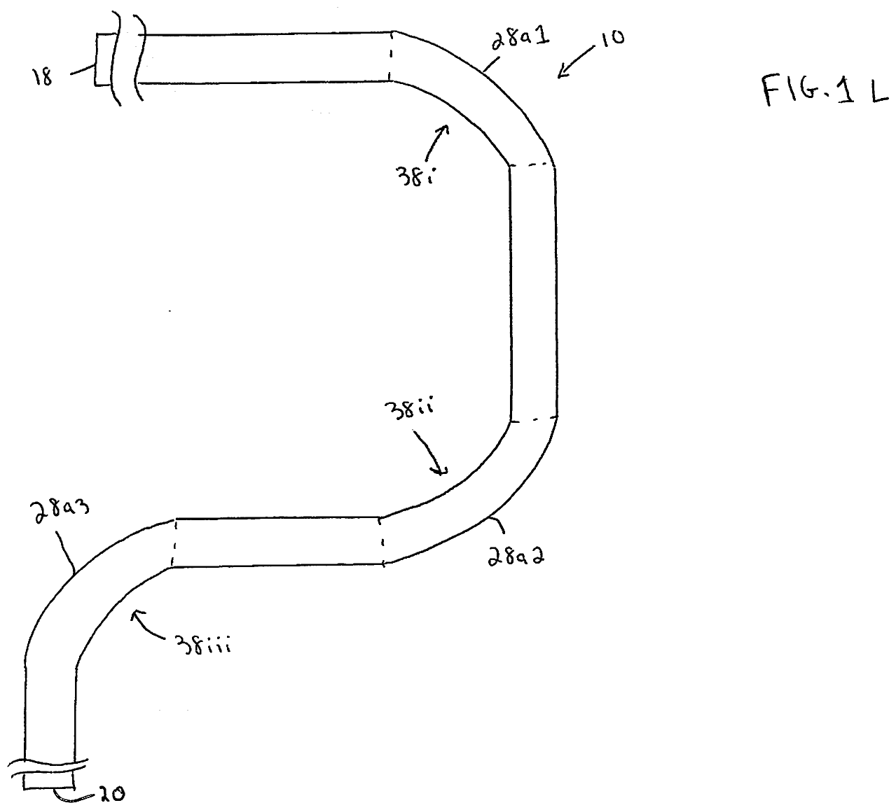

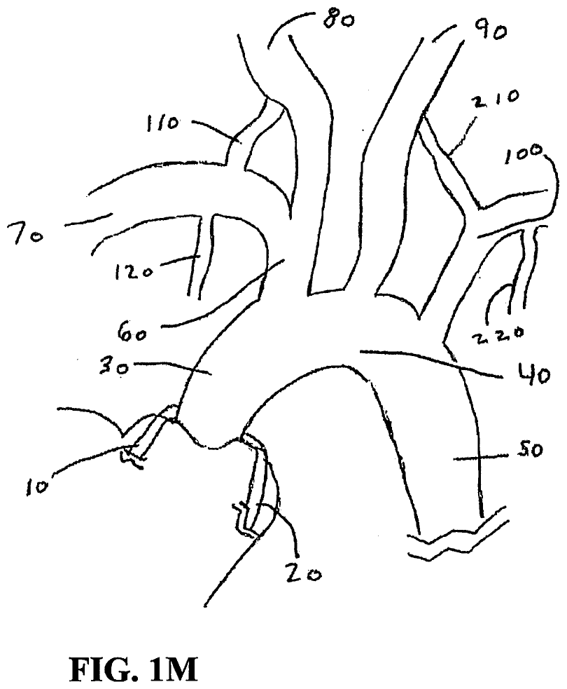

FIG. 1A illustrates a medical device 10 (e.g., a catheter 12) according to the principles of the present disclosure. The medical device 10 includes a body 14 defining a longitudinal axis X and having an outer wall 16; a proximal end hole 18; and a distal end hole 20 that is positioned opposite to the proximal end hole 18. The body 14 of the medical device 10 defines an overall length L that lies substantially within the range of approximately 10 cm to approximately 180 cm and an external diameter D that lies substantially within the range of approximately 0.5 Fr to approximately 35 Fr. The body 14 includes at least one working lumen 22 which, in the particular embodiments shown throughout the figures, extends centrally through the body 14 from the proximal end hole 18 to the distal end hole 20 and provides the sole working channel through the medical device 10. The working lumen thus acts as a conduit through which additional catheters, wires, interventional devices, and other endovascular devices may be delivered to a target site as well as locations more distal within the patient's vasculature.

As seen in FIG. 1A, the medical device 10 may include an external termination device 24 (e.g., a Luer lock 26). It is also envisioned that the medical device 10 may include a diaphragm.

The medical device 10 includes a plurality of segments 28 and a plurality of pull wires 30. More specifically, the medical device 10 includes a plurality of inactive (passive) segments 28i and a plurality of active (steerable, deflectable, articulable) segments 28a that are spaced along the longitudinal axis X and connected to the plurality of pull wires 30. The inactive segments 28i and the active segments 28a are arranged in a staggered pattern such the body 14 alternates between inactive segments 28i and active segments 28a.

In the embodiment seen in FIG. 1A, for example, each active segment 28a is connected to a corresponding (single) pull wire 30 that extends through (within/embedded in) the outer wall 16 of the body 14 such that the number of pull wires 30 corresponds to the number of active segments 28a. Upon the application of an axial (pulling) force to each of the pull wires 30, the corresponding active segment 28a is deflected (articulated) to thereby reconfigure (actively steer) the medical device 10 between a first (initial, normal) configuration (FIG. 1A), in which the body 14 is (generally) linear in configuration, and a second (subsequent, deflected) configuration (FIG. 1C), in which the body 14 is non-linear in configuration. As described in further detail herein, reconfiguration of the body 14 (and the medical device 10) between the first and second configurations not only facilitates access to various locations within the patient's vasculature, but allows for anchoring (bracing) of the medical device 10 within the patient's vasculature.

The use of a single pull wire 30 in connection with each active segment 28a reduces the requisite number of pull wires 30, thus reducing complexity in both construction and operation of the medical device 10. In the embodiment seen in FIG. 1A, for example, each pull wire 30 is received within a corresponding channel 32 that extends through the outer wall 16 of the body in (generally) parallel relation to the working lumen 22 such that the pull wires 30 are embedded within the medical device 10.

To facilitate the application of axial force to the pull wires 30, in certain embodiments, the medical device 10 may include a plurality of corresponding activating mechanisms 34 (e.g., such that the number of pull wires 30 corresponds to the number of activating mechanisms 34). In the embodiment seen in FIG. 1A, for example, the medical device 10 includes a (first) activating mechanism 34i that is connected to the pull wire 30i and a (second) activating mechanism 34ii that is connected to the pull wire 30ii. The activating mechanisms 34 may include any structure or mechanism suitable for the intended purpose of applying the axial force to the pull wires 30 required to deflect the medical device 10 as necessary or desired, such as, for example, rotating wheels, pulley systems, or the like. In certain embodiments, it is envisioned that the active segments 28a, the pull wires 30, and the activating mechanisms 34 may be configured (and connected) such that each pull wire 30 may be individually acted upon to deflect (steer) the corresponding segment 28a in a single direction only.

In the embodiment seen in FIG. 1A, for example, the medical device 10 includes a first inactive segment 28i1; a first active segment 28a1 (also referred to herein as the “secondary steering segment”) that is located distally of the segment 28i1; a second inactive segment 2812 that is located distally of the segment 28a1; and a second active segment 28a2 (also referred to herein as the “primary steering segment”) that is located distally of the segment 2812. Additionally, the device includes respective first and second pull wires 30i, 30ii that are located within the channel 32, as seen in FIG. 1B. It is also envisioned, however, that the first and second pull wires 30i, 30ii may be located within separate channels 32i, 32ii (e.g., such that the number of channels 32 corresponds to the number of pull wires 30), as seen in FIG. 1D. The channels 32i,32ii are shown embedded in the wall of the device.

The pull wires 30i, 30ii are connected to the segments 28a1, 28a2 at connection points 36i, 36ii (in addition to the activating mechanism 34i, 34ii), respectively, so as to facilitate reconfiguration of the medical device 10 between the first configuration (FIG. 1A) and the second configuration (FIG. 1C). More specifically, upon reconfiguration of the medical device 10, the active segments 28ai, 28aii define respective first and second bends 38i, 38ii, which may be either substantially similar (e.g., identical) or dissimilar depending, for example, upon the particular configuration of the segments 28a1, 28a2, the materials of construction used in the body 14, the particular requirements of the catheter 12 dictated by the medical procedure, etc. Although the bends 38i, 38ii are each illustrated as being (approximately) equal to 90 degrees in FIG. 1C, depending upon the particular configuration of the segments 28a1, 28a2, the requirements of the trans-radial access procedure, the particular anatomy of the patient's vasculature, etc., it is envisioned that the bends 38i, 38ii may lie substantially within the range of approximately 0 degrees to approximately 270 degrees. For example, in one particular embodiment, it is envisioned that the segment 28a1 may be configured such that the bend 38i lies substantially within the range of approximately 0 degrees to approximately 180 degrees (e.g., approximately 90 degrees to approximately 180 degrees) and that the segment 28a2 may be configured such that the bend 382 lies substantially within the range of approximately 0 degrees to approximately 270 degrees (e.g., approximately 90 degrees to approximately 270 degrees).

In the particular embodiment of the present disclosure seen in FIG. 1A, for example, the connection points 36i, 36ii are shown as being in (general) angular alignment (e.g., along a circumference of the body 14 of the medical device 10), which facilitates deflection of the segments 28a1, 28a2 in similar (e.g., identical) directions, as seen in FIG. 1C. It is also envisioned, however, that the connection points 36i, 36ii may be angularly offset so as to facilitate deflection of the segments 28a1, 28a2 in dissimilar directions. For example, with reference to FIGS. 1E and 1F, it is envisioned that the channels 32i, 32ii and the connection points 36i, 36ii may be oriented in (generally) diametric opposition such that the bends 38i, 38ii respectively defined by the segments 28a1, 28a2d curve in (generally) opposite directions.

With reference again to FIG. 1A, in certain embodiments, the medical device 10 may include an inflatable balloon 40 that is supported on the body 14 proximally of the distal end hole 20. While the medical device 10 is illustrated as including a single inflatable balloon 40 including a circumferential configuration that completely circumscribes the body 14, it should be appreciated that the configuration of the inflatable balloon 40 and the particular number of inflatable balloons 40 may be altered in various embodiments without departing from the scope of the present disclosure. For example, it is envisioned that the medical device 10 may include a plurality of circumferential or non-circumferential inflatable balloons 40, which may be located at different locations along the longitudinal axis X. Such foregoing alternatives are applicable to each of the devices described herein.

To facilitation inflation of the inflatable balloon(s) 40, the medical device 10 is configured for connection to an inflation source 42 (FIG. 1A). It is envisioned that fluid from the inflation source 42 may be communicated to the inflatable balloon(s) 40 in any suitable manner. For example, the medical device 10 may include one or more fluid conduits 44 (FIG. 1B) extending through the outer wall 16 of the body in (generally) parallel relation to the working lumen 22 (and the channel 32). In such embodiments, the fluid conduit(s) 44 are configured to establish fluid communication between the inflation source 42 and the inflatable balloon 40 such that fluid is communicable from the inflation source 42, through the fluid conduit(s) 44, and to the inflatable balloon 40 (during inflation) and from the inflatable balloon 40, through the fluid conduit(s) 44, and to the inflation source 42 (during deflation). Although shown as including a single fluid conduit 44, it should be appreciated that the particular number (and/or the orientation) of fluid conduits 44 included in the medical device 10 may be altered without departing from the scope of the present disclosure. The devices can include a side port for communication with the inflation source.

FIGS. 1G-1K illustrate another embodiment of the medical device 10, which is referred to by the reference character 50. The device 50 is substantially similar to the aforedescribed medical device 10 and, as such, in the interest of brevity, will only be discussed with respect to any differences therefrom.

The device 50 includes a plurality of inactive segments 5211, 5212 and a plurality of active segments 52a1, 52a2 that are connected to a plurality of pull wires 54. In contrast to the medical device 10, one or more of the active segments 52a is connected to a pair of pull wires 54 (rather than a single pull wire, as discussed above in connection with the medical device 10). For example, in the configuration seen in FIG. 1G, the device 50 includes (first and second) pull wires 54i, 54ii that are connected to the segment 52a1 and a (third) pull wire 54iii that is connected to the active segment 52a2. FIG. 1H illustrates an alternate configuration in which the pull wires 54i, 54ii are connected to the segment 52a2 and the pull wire 54iii is connected to the active segment 52al.

The pull wires 54i, 54iii are located within a common channel 56i (FIG. 1I) and the pull wire 54ii is located within a channel 56ii, each of which extends through an outer wall 58 of the device 50. It is also envisioned, however, that each of pull wires 54i, 54iii may be accommodated within a dedicated channel 56. The pull wires 54i, 54ii are connected to the segment 52a1 at connection points 60i, 60ii that are located in (generally) diametric opposition and to activating mechanisms 62i, 62ii, respectively, which allows for deflection of the segment 52a1 in multiple directions (e.g., first and second opposing directions). The pull wire 54iii is connected to the segment 52a2 at a connection point 60iiii and to activating mechanisms 62iiii, which allows for deflection of the segment 52a2 in a single direction only.

FIG. 1J illustrates an alternate configuration of the device 50 including a (fourth) pull wire 54iv that is connected to the segment 52a2 such that each of the segments 52a1, 52a2 is connected to a pair of pull wires 54.

The pull wires 54ii, 54iv are (commonly) located within the channel 56ii and the pull wires 54i, 54iii are (commonly) located within the channel 56i. It is also envisioned, however, that each of pull wires 54i, 54ii, 54iii, 54iv may be accommodated within a dedicated channel 56. The pull wires 54i, 54ii are connected to the segment 52a1 at connection points 60i, 60ii that are located in (generally) diametric opposition and to activating mechanisms 62i, 62ii, respectively, which allows for deflection of the segment 52a2 in multiple directions (e.g., first and second opposing directions). Similarly, the pull wires 54iii, 54iv are connected to the segment 52a2 at connection points 60iii, 60iv that are located in (generally) diametric opposition and to activating mechanisms 62iii, 62iv, respectively, which allows for deflection of the segment 52a2 in multiple directions (e.g., first and second opposing directions).

With reference again to FIG. 1A, for example, in certain embodiments, the medical device 10 may include one or more additional pull wires 30 that may be embedded within one or more additional channels 32 extending through the outer wall 16 of the medical device 10. In such embodiments, these additional channels 32 may not only accommodate the additional pull wires 30, but may be configured as fluid conduits to allow for the communicate of inflation fluid therethrough (e.g., from the inflation source 42) to facilitate inflation and deflation of the inflatable balloon 40. For example, it is envisioned that the devices described herein may include a first plurality of lumens (that extend through the outer wall of the device) that are configured to accommodate pull wires and one or more second lumens that are devoid of pull wires such that the second lumen(s) acts solely as fluid conduits that are configured to facilitate inflation of the inflatable balloon via the communication of fluid therethrough. In such embodiments, the lumen(s) (conduit(s)) used for inflation and deflation of the balloon may be disposed substantially within the outer wall of the device within an effective segment thereof, which refers to that section of the device that is positioned within the patient's vasculature. In certain embodiments, it is envisioned that the lumen(s) (conduit(s)) used for inflation and deflation of the balloon may branch away from the outer wall of the device along a proximal portion of the device externally of the patient.

As mentioned above, it is envisioned that the medical device 10 may include a single (circumferential) inflatable balloon 40 (e.g., located at, adjacent to, or near the distal end hole 20). When inflated, it is envisioned that the inflatable balloon 40 may be configured to alter flow in the vessel and/or anchor the medical device 10 within the patient's vasculature. Some embodiments may include more than one inflatable balloon 40 and/or at least one irrigation channel 46, as seen in FIG. 1B. In such embodiments, the irrigation lumen 46 may extend substantially within the outer wall 16 of the medical device 10 (e.g., along the effective segment). It is envisioned that one or more of the channels 32, 46 extending within the outer wall 16 of the medical device 10 may exit the medical device 10 and branch off at locations proximal to the percutaneous access site.

In various embodiments of the present disclosure, the devices described herein may include a hydrogel element either in addition to or in place of any inflatable balloon(s) 40. In such embodiments, it is envisioned that the hydrogel element(s) may function as balloons by hydrating or dehydrating in the presence of blood/fluid and/or in response to an additional stimulus, to thereby alter flow within the vasculature and/or anchor the device.

As indicated above, the devices described herein include at least two steering segments: the active (primary steering) segment 28a2 and the active (secondary steering) segment 28a1. In certain embodiments, it is envisioned that the segment 28a2 may be located along any desired segment of the body 14 from the distal end hole 20 to a point that is located up to approximately 8 cm proximal to the distal end hole 20. In other words, it is envisioned that the segment 28a2 may define a length L2 that lies substantially within the range of approximately 0.1 cm to approximately 7 cm. As mentioned above, it is envisioned that the segment 28a2 may be capable of being actively curved/bent (e.g., via a pulley effect on the pulley wire(s) connected thereto) from 0 degrees to approximately 180 degrees, any such bend being referring to herein as an “after-bend,” as defined further below.

As indicated above, the active (secondary steering) segment 28a1 is located proximally of the active segment 28a2 (e.g., along any secondary segment of the body 14 of the catheter 12). In certain embodiments, it is envisioned that the segment 28a1 may space a distance from the distal end hole 20 that lies substantially within the range of approximately 2 cm to approximately 30 cm. In other words, it is envisioned that the segment 28a1 may define a length L1 that lies substantially within the range of approximately 0.4 cm to approximately 15 cm. As mentioned above, it is envisioned that the segment 28a1 may be capable of being actively curved/bent (e.g., via a pulley effect on the pulley wire(s) connected thereto) from 0 degrees to approximately 270 degrees. It is further envisioned that any such (secondary) curve/bend may be on the same side of the body 14 and that the (primary) curve of the active segment 28a2 and the (secondary) curve of the active segment 28a1 may be substantially similar (e.g., identical) or dissimilar.

In alternative embodiments of the present disclosure, it is envisioned that any of the aforedescribed devices may include at least one additional pull wire to create at least one additional active steering segment capable of creating at least one additional curve/bend. It is also envisioned that the pull wires may be oriented in different directions and that steering segments may overlap along a particular section (length) of the medical device 10.

In one particular embodiment, which is seen in FIG. 1L, the medical device 10 includes a (third) inactive segment 28i3 and a (third) active segment 28a3 that defines a (tertiary) bend 38iii upon deflection. For example, it is envisioned that the active segment 28a3 may be located along a segment that is at least 0.4 cm long and that is located up to 8 cm from the distal end hole 20. In the particular embodiment illustrated in FIG. 1L, whereas the bends 38i, 38ii curve in a first direction, the bend 38iii curves in a second direction (e.g., (generally) opposite to the first direction). The bends 38i, 38ii, 38iii may be used through an external sheath, or “bare-back” (e.g., without any such external sheath).

In some embodiments, such as those including the aforementioned inflatable balloon 40, a “peel-away” sheath 48 (FIG. 1A) may be used to facilitate insertion of the medical device 10 (e.g., the section of the medical device 10 that includes the inflatable balloon 40), which can be removed and peeled away, and remainder of the medical device 10 can be used “bare-back” when desired. In some alternate versions the sheath 48 may also be configured to serve as an introducer into an outer sheath as well.

It is envisioned that any of the devices described herein (e.g., the aforedescribed devices 10, 50) may be configured for use with a removable inner dilator 49, as seen in FIG. 1A, which is configured for insertion into the medical device 10, for example, to aid with insertion and reduce any “shelf” between the catheter 12 and any insertion wire. In such embodiments, the inner dilator 49 includes an outer diameter that is less than an inner diameter defined by the body 14 and a length that is greater than the length L defined by the body 14. The dilator 49 can include a distal end hole (that can extend past the distal end hole of the sheath) and a proximal hole.

In many cases, especially diagnostic angiograms, the device according to the present disclosure may be the only catheter used. In other cases, including many but not all diagnostic angiograms and many but not all interventions, the device according to the present disclosure may be used with at least one supplemental (additional) device (e.g., a catheter, a wire, etc.) that is passed therethrough and into the patient's vasculature (e.g., to access more distal locations). During such use, it is envisioned that the device according to the present disclosure (and the various bends/curves described herein) may aid in appropriate positioning and directional assistance for advancing such supplemental devices secondary inner structures. It is also envisioned that when the devices according to the present disclosure are in the second (subsequent, deflected) configuration (FIG. 1C), the non-linear configuration thereof may inhibit (if not entirely prevent) kickback and prolapse that may otherwise occur as the supplemental device(s) are advanced distally therethrough into the patient's vasculature. Thus, the device can provide in some embodiments added support for advancement of the inner wires/catheters/devices by two mechanisms—1) the wires themselves when shortened to produce a curve will resist straightening and therefore also resist catheter displacement, kickback and prolapse out of the target vessel; and 2) use the lesser curve of the aortic arch and rest one of the catheter curves against the curve for added support.

In various embodiments, the device according to the present disclosure may be configured for engagement (contact) with a vascular arch (e.g., the lesser curve of the aortic arch) to enhance use of the device via support (bracing) against the vascular arch. It is envisioned that bracing the device against (e.g., along) the vascular arch may provide further additional support for the device to further inhibit (if not entirely prevent) kickback and prolapse, as previously described in a prior patent by Walzman, specifically, U.S. patent application Ser. No. 16/290,923, filed 3 Mar. 2019; and U.S. Pat. No. 10,258,371, issued 16 Apr. 2019.

In many diagnostic and interventional procedures, the device according to the present disclosure can act as the sole “guide” catheter and may support the insertion of additional medical devices therethrough. Alternatively, in other interventional cases, an additional inner “guide” catheter can be passed through the device according to the present disclosure and more distally in the vasculature (e.g., in order to accommodate additional medical devices therethrough). In still other procedures the device according to the present disclosure can be used with or without additional inner wires and/or catheters (e.g., in order to optimally place an “exchange wire” and/or other similar exchange device into a target area, as facilitated by the steering and bending capabilities of the device), and the device according to the present disclosure can then be exchanged out for and replaced with a different catheter which will advance over said wire and/or other exchange device. Thereby in some cases a catheter with a larger inner diameter can be used for a corresponding outer diameter, allowing delivery of still more additional interventional devices for a given size of a patient's radial artery. Since the device according to the present disclosure has at least two pull wires (e.g., located substantially within the outer wall of the body), catheters without any such pull wires can be made with thinner walls and can thereby have a larger maximal inner diameter for a given outer diameter. In various cases, various embodiments of the device according to the present disclosure with various segment lengths can be chosen, depending on the desired region(s) to access and in the patient's particular anatomy.

FIG. 1M illustrates the vessels proximal to the aorta, including the right coronary artery 10, the left coronary artery 20, ascending aorta 30, arch of aorta 40, descending aorta 50, brachiocephalic artery 60, right subclavian artery 70, right common carotid artery 80, left common carotid artery 90, and left subclavian artery 100. Additionally, illustrated are the right vertebral artery 110, the right internal mammary artery 120, left vertebral artery 210 and left internal mammary artery 220.

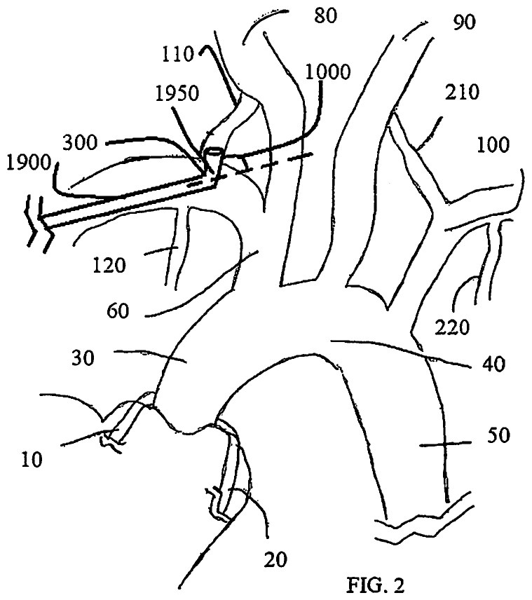

FIG. 2 illustrates one embodiment of the device according to the present disclosure during the right radial approach to the right vertebral artery 110. The illustrated device may be substantially similar (or identical) to any of the aforedescribed devices (e.g., the devices 10, 50) and includes an after-bend angle 1000 that is defined as the angle between a segment 1900 of the device before a bend/curve 300 and a segment 1950 of the device after the bend/curve 300. The dashed lines indicate the direction of the segment 1950 prior to deflection of the device.

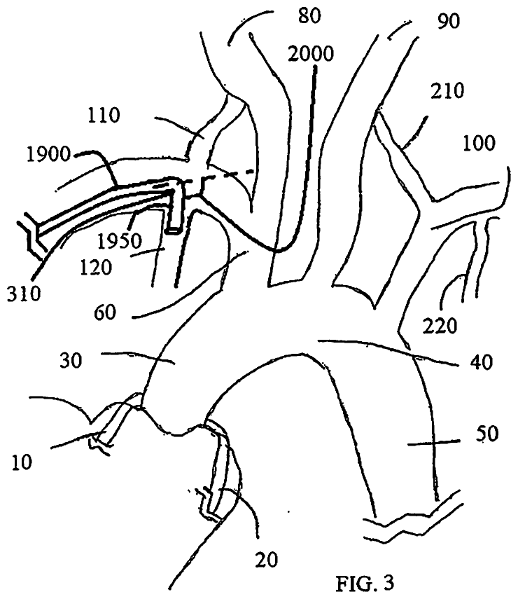

FIG. 3 illustrates use of the device during the right radial approach to the right internal mammary artery 120. In FIG. 3, the device includes an after-bend angle 2000 that is defined as the angle between the segment 1900 of the device before a bend/curve 310 and the segment 1950 after the bend/curve 310. The dashed lines indicate the direction of the segment 1950 of the catheter prior to deflection. The bend/curve 310 may embody the aforementioned tertiary bend or, alternatively, may represent the primary bend when the catheter is rotated within the vasculature.

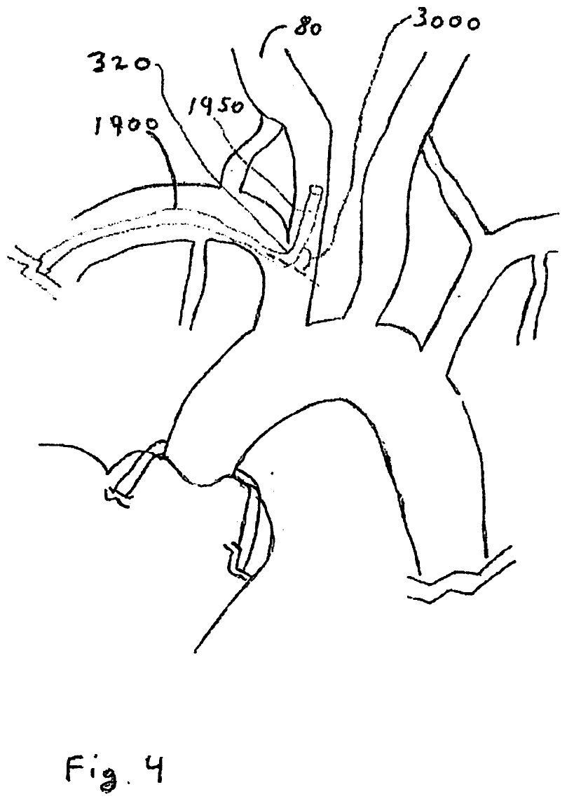

FIG. 4 illustrates use of the device during the right radial approach to the right common carotid artery 80. In FIG. 4, the device includes an after-bend angle 3000 that is defined between the segment 1900 of the device before a bend/curve 320 and the segment 1950 after the bend/curve 320. The dashed lines indicate the direction of the segment 1950 of the catheter prior to deflection. In the illustrated method of use (embodiment), the after-bend angle 3000 constitutes the primary curve/bend.

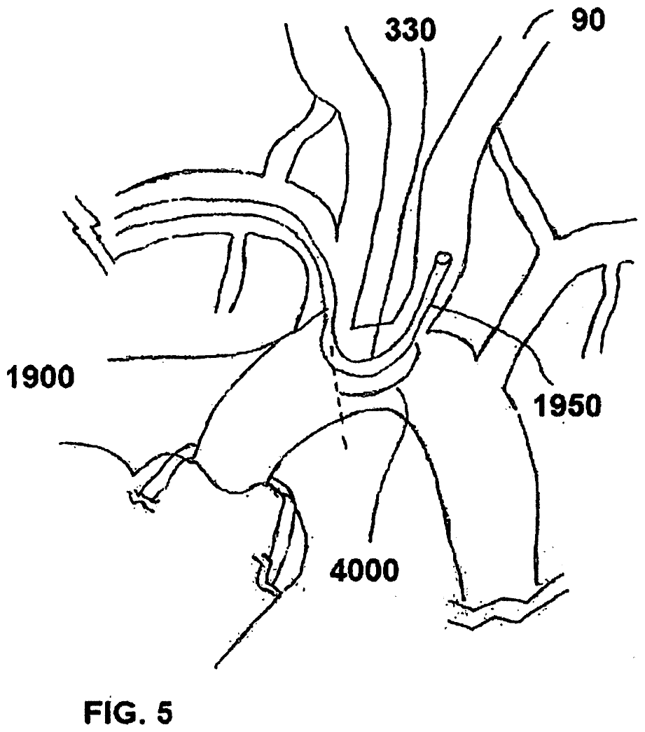

FIG. 5 illustrates use of the device during the right radial approach to the left common carotid artery 90. In FIG. 5, the device includes an after-bend angle 4000 that is defined between the segment 1900 of the device before a bend/curve 330 and the segment 1950 after the bend/curve 330. The dashed lines indicate the direction of the segment 1950 of the catheter before deflection.

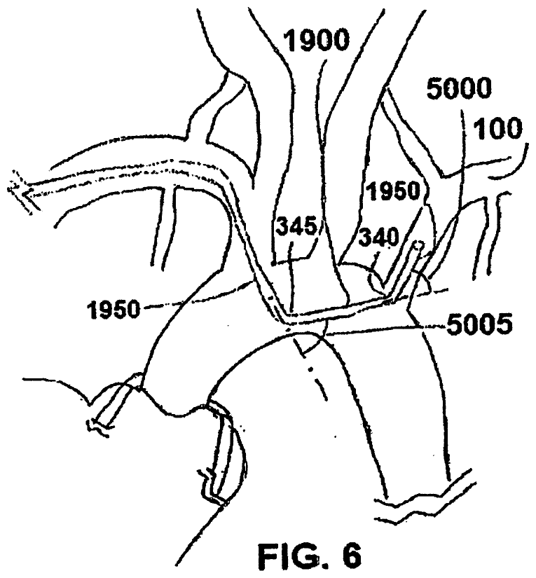

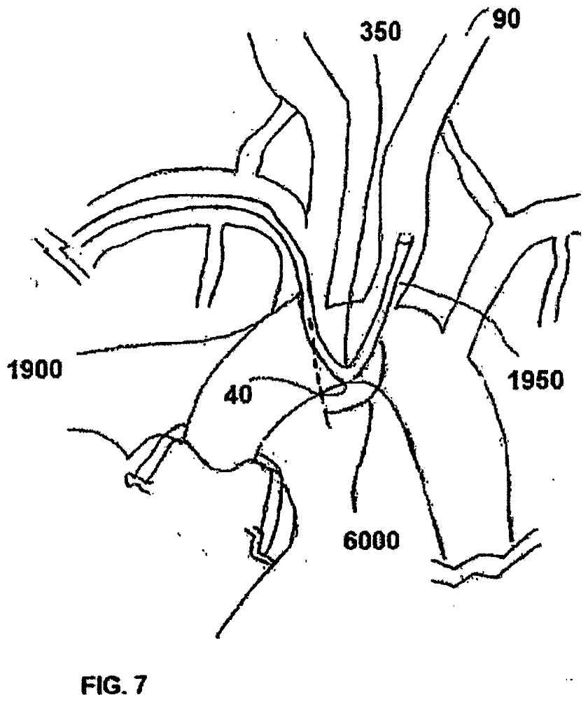

FIG. 6 illustrates use of the device during the right radial approach to the left subclavian artery 100, which is another technique according to the principles of the present disclosure. In FIG. 6, the device includes a (primary) after bend angle 5000 that is defined between the segment 1900 of the device before a (primary) bend/curve 340 and the segment 1950 of the catheter after the bend/curve 340. The dashed lines indicate the direction of the segment 1950 of the catheter before deflection. In the illustrated method of use (embodiment), the device also includes a (secondary) after bend angle 5005 that is defined between the segment 1950 before a (secondary) bend/curve 345 and the segment 1900 after the bend/curve 345. The combination of dashed and dotted lines indicates the directions of the segment 1950 before deflection. In some embodiments by way of example, the proximal bend zone adjacent the aortic arch can have a length of approximately 40 mm, the straight segment (section) extending in the left subclavian artery can have a length of 40 mm and the distal bend zone within the left subclavian artery can have a length of 20 mm. The distal and proximal bend can be in the same plane.

FIG. 7 illustrates use of the device during the right radial approach to the left common carotid artery 90 and the implementation of an arch fulcrum support technique (configuration). In FIG. 7, the device includes an after-bend angle 6000 that is defined between the segment 1900 of the device before a bend/curve 350 and the segment 1950 of the catheter after the bend/curve 350. FIG. 7 illustrates an alternate technique of using the device(s) described herein in which the device is positioned against the arch fulcrum 40, thereby using the Walzman arch fulcrum support technique, as described in U.S. Pat. No. 10,258,371, for example. It should be understood that the same device(s) and technique(s) can also be used via a left radial approach.

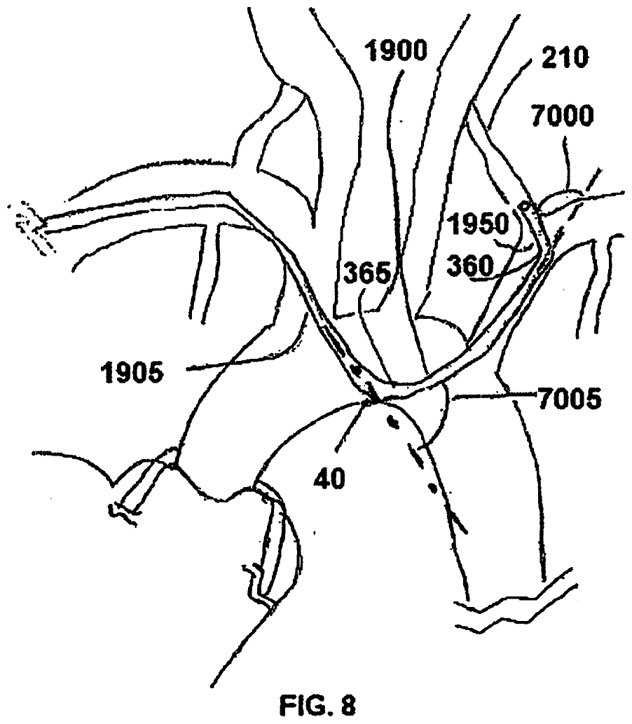

FIG. 8 illustrates use of the device during the right radial approach to the left vertebral artery 210 and the implementation of the arch fulcrum support technique (configuration). In FIG. 8, the device includes a (primary) after bend angle 7000 that is defined between the segment 1900 of the device before a bend/curve 360 and the segment 1950 of the device after the bend/curve 360. The dashed lines indicate the direction of the catheter element 1900 before the catheter bend/curve 360. In the illustrated method of use (embodiment), the device also includes a (secondary) after bend angle 7005 that is defined between the segment 1950 before the bend/curve 365 and the segment 1900 of the device after the catheter bend/curve 365. The combination of dashed and dotted lines indicates the direction of the segment 1950 before deflection. As seen in FIG. 8, during the illustrated procedure, the device rests on the arch fulcrum 40, thereby using the Walzman arch fulcrum support technique.

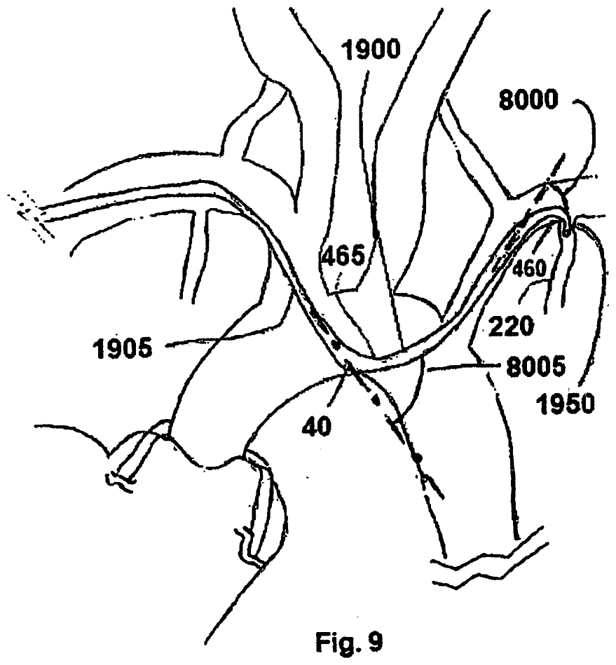

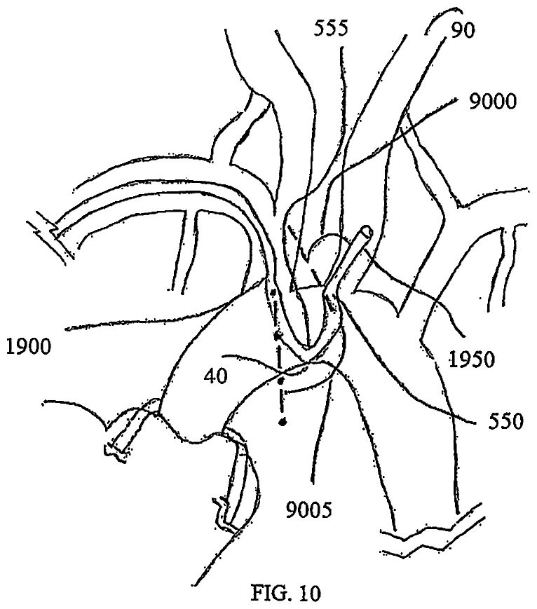

FIG. 9 illustrates use of the device during the right radial approach to the internal mammary artery 220. In FIG. 9, the device includes a (primary) after bend angle 8000 that is defined between the segment 1900 of the device before a bend/curve 460 and the segment 1950 of the device after the bend/curve 460. The dashed lines indicate the direction of the segment 1950 before deflection. In the illustrated method of use (embodiment), the device also includes a (secondary) after bend angle 8005 that is defined between the segment 1950 before the bend/curve 465 and the segment 1900 of the device after the bend/curve 465. The combination of dashed and dotted lines indicates the direction of the segment 1950 before deflection. As seen in FIG. 9, during the illustrated procedure, the device rests on the arch fulcrum 40, thereby using the Walzman arch fulcrum support technique.

FIG. 10 illustrates use of the device during the right radial approach to the left common carotid artery 90 and the implementation of the arch fulcrum support technique (configuration). In FIG. 10, the device includes a (primary) after bend angle 9000 that is defined between the segment 1900 of the device before a bend/curve 550 and the segment 1950 of the device after the bend/curve 550. The dashed lines indicate the direction of the segment 1950 before deflection. In the illustrated method of use (embodiment), the device also includes a (secondary) after bend angle 9005 that is defined between the segment 1950 before the bend/curve 555 and the segment 1900 after the bend/curve 555. The combination of dashed and dotted lines indicates the direction of the segment 1950 before deflection. As seen in FIG. 9, during the illustrated procedure, the device rests on the arch fulcrum 40, thereby using the Walzman arch fulcrum support technique.

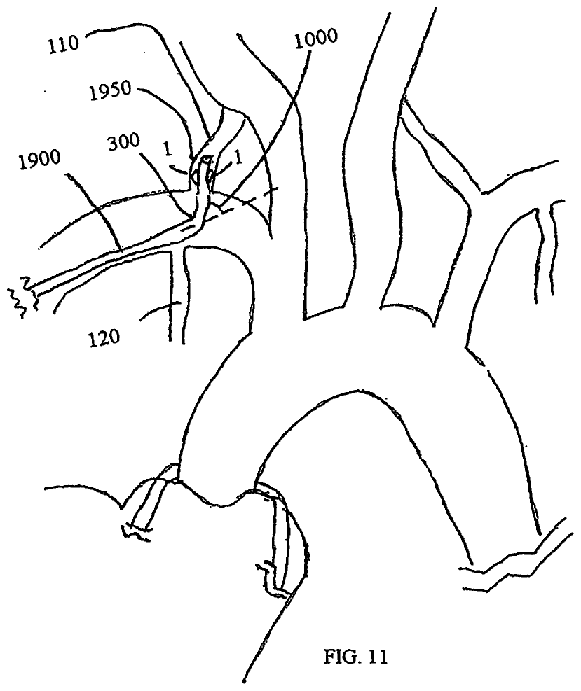

FIG. 11 illustrates use of the device during the procedure illustrated in FIG. 2. The device, however, includes a (partially inflated) balloon 1 (or hydrogel) (which may be substantially similar (e.g., identical) to the aforedescribed inflatable balloon 40 or hydrogel).

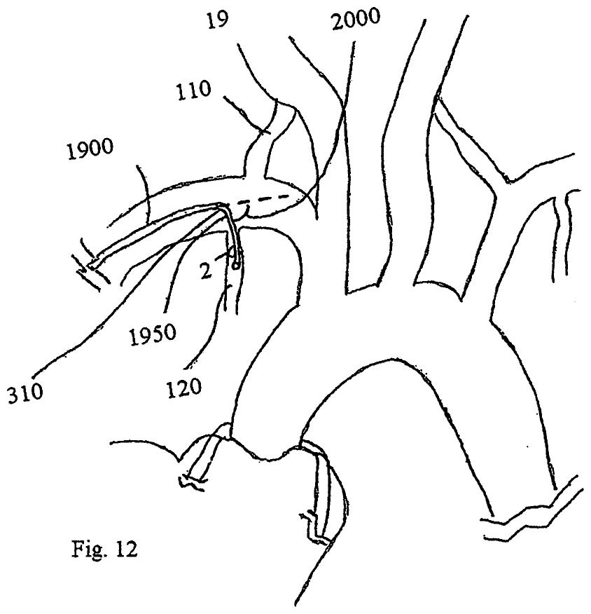

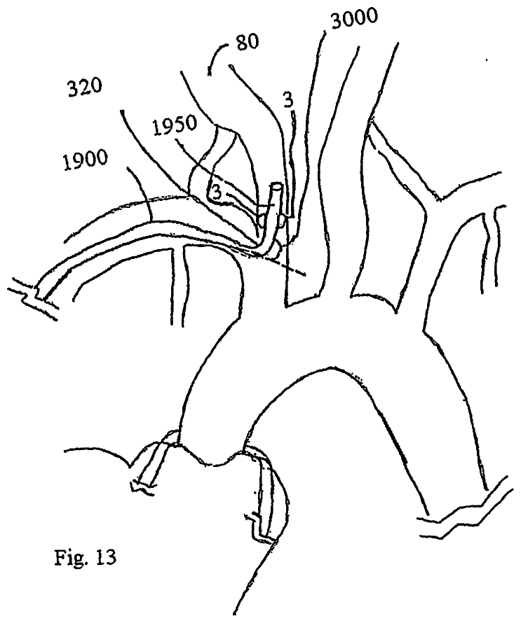

FIG. 12 illustrates use of the device during the procedure illustrated in FIG. 3. The device, however, includes a (partially inflated) balloon 2 (or hydrogel) (which may be substantially similar (e.g., identical) to the aforedescribed inflatable balloon 40 or hydrogel).

FIG. 13 illustrates use of the device during the procedure illustrated in FIG. 4. The device, however, includes a (partially inflated) balloon 3 (or hydrogel) (which may be substantially similar (e.g., identical) to the aforedescribed inflatable balloon 40 or hydrogel).

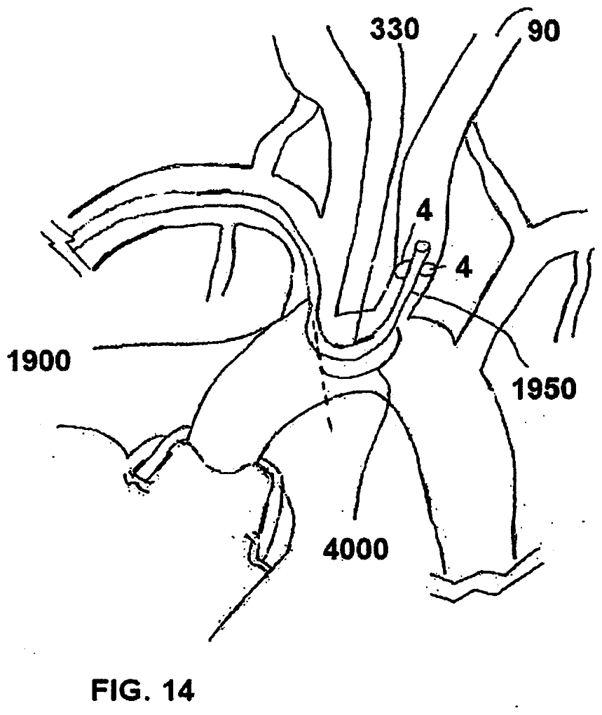

FIG. 14 illustrates use of the device during the procedure illustrated in FIG. 5. The device, however, includes a (partially inflated) balloon 4 (or hydrogel) (which may be substantially similar (e.g., identical) to the aforedescribed inflatable balloon 40 or hydrogel).

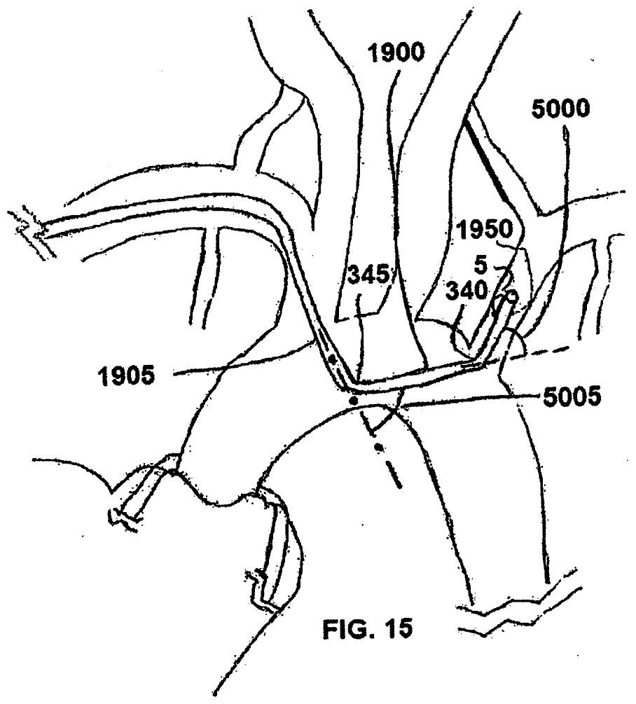

FIG. 15 illustrates use of the device during the procedure illustrated in FIG. 6. The device, however, includes a (partially inflated) balloon 5 (or hydrogel) (which may be substantially similar (e.g., identical) to the aforedescribed inflatable balloon 40 or hydrogel).

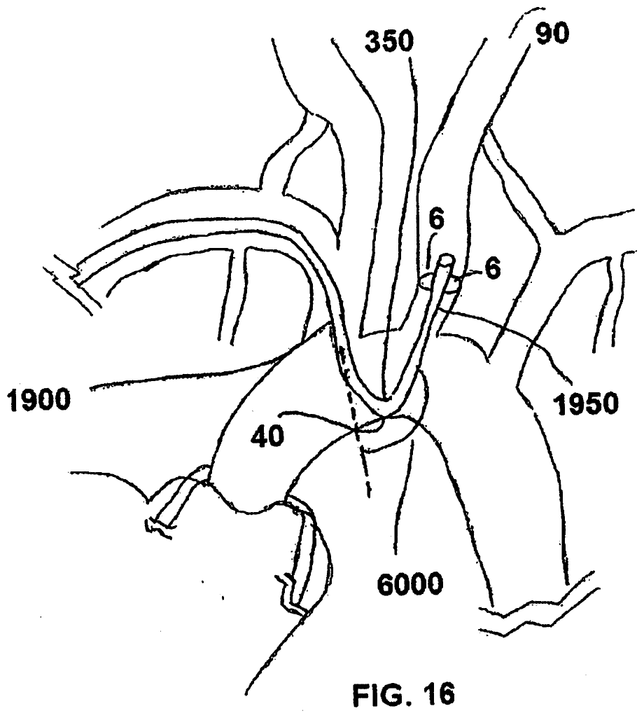

FIG. 16 illustrates use of the device during the procedure illustrated in FIG. 7. The device, however, includes a (partially inflated) balloon 6 (or hydrogel) (which may be substantially similar (e.g., identical) to the aforedescribed inflatable balloon 40 or hydrogel).

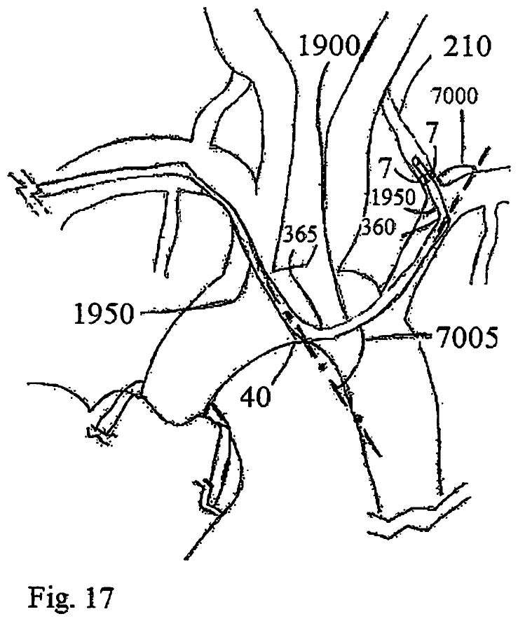

FIG. 17 illustrates use of the device during the procedure illustrated in FIG. 8. The device, however, includes a (partially inflated) balloon 7 (or hydrogel) (which may be substantially similar (e.g., identical) to the aforedescribed inflatable balloon 40 or hydrogel).

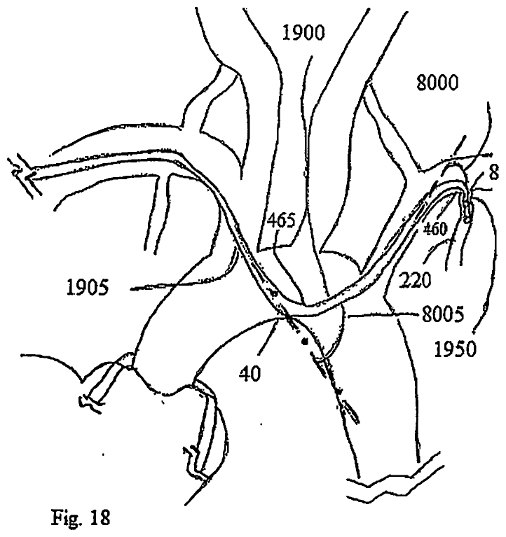

FIG. 18 illustrates use of the device during the procedure illustrated in FIG. 9. The device, however, includes a (partially inflated) balloon 8 (or hydrogel) (which may be substantially similar (e.g., identical) to the aforedescribed inflatable balloon 40 or hydrogel).

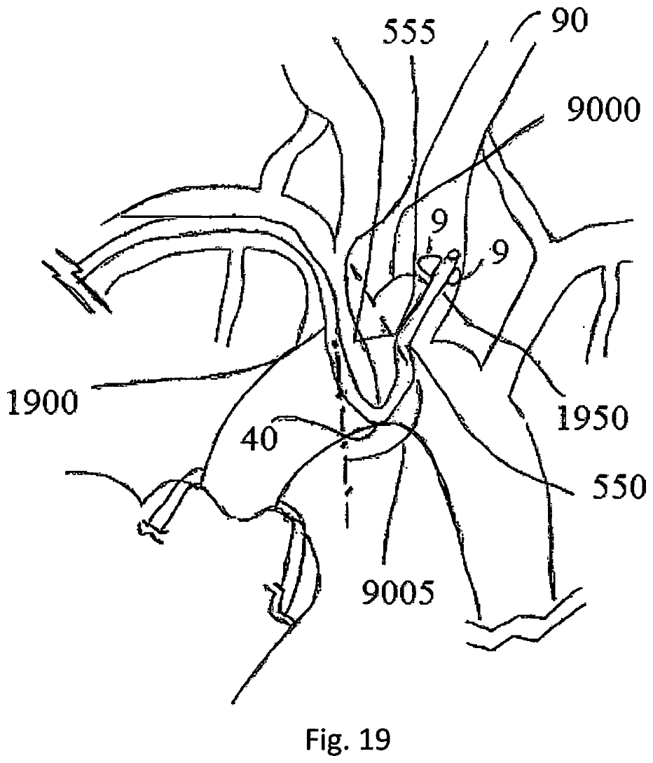

FIG. 19 illustrates use of the device during the procedure illustrated in FIG. 10. The device, however, includes a (partially inflated) balloon 9 (or hydrogel) (which may be substantially similar (e.g., identical) to the aforedescribed inflatable balloon 40 or hydrogel).

It should be noted that the prior art has variously defined calculations of bend angles from an inner curve or outer curve perspective. Additionally, prior art references are often ambiguous as to which measurement perspective is being described. Said angles described herein is a measured starting from a line drawn straight from the catheter before a bend/curve and extending straight beyond said bend/curve. This disclosure, therefore, includes sample bend angulation notations to optimally describe the angles referred to herein. Still further, the drawings associated with the present disclosure depict a right radial approach but left radial approach can also be used. Additional approaches and device uses are optionally possible as well. The devices are also optimally designed for percutaneous use. Notwithstanding this, uses via other approaches, including those that are not percutaneous, are possible as well.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

Any publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

Additionally, persons skilled in the art will understand that the elements and features shown or described in connection with one embodiment may be combined with those of another embodiment without departing from the scope of the present disclosure and will appreciate further features and advantages of the presently disclosed subject matter based on the description provided.

Throughout the present disclosure, terms such as “approximately,” “generally,” “substantially,” and the like should be understood to allow for variations in any numerical range or concept with which they are associated. For example, it is intended that the use of terms such as “approximately” and “generally” should be understood to encompass variations on the order of 25% (e.g., to allow for manufacturing tolerances and/or deviations in design).

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

1-20. (canceled)

21. A method of establishing trans-radial access to a vascular target site in a patient, the method comprising:
introducing a medical device via a percutaneous opening into the patient's right radial artery, wherein the medical device includes:
a proximal end;
a distal end;
at least one steerable segment; and
at least one pull wire connected to the at least one steerable segment to thereby facilitate reconfiguration of the medical device between a normal configuration, in which the medical device is generally linear in configuration, and a deflected configuration, in which the at least one steerable segment of the medical device is non-linear in configuration;
advancing the distal end of the medical device into the patient's right subclavian artery; and
advancing an inner catheter through the medical device and into the patient's right vertebral artery.

22. The method of claim 21, further comprising:
applying force to a first pull wire to reconfigure the medical device from the normal configuration into the deflected configuration and thereby steer the distal end of the medical device into or towards the patient's right vertebral artery.

23. The method of claim 22, further comprising:
withdrawing the inner catheter;
returning the medical device to the normal configuration;
advancing the distal end of the medical device into the patient's innominate artery; and
applying force to a second pull wire to reconfigure the medical device from the normal configuration into deflected configuration and thereby steer the distal end of the medical device into the patient's right common carotid artery.

24. The method of claim 23, further comprising:
advancing the inner catheter through the medical device and into the patient's right internal carotid artery or the patient's right external carotid artery.

25. The method of claim 24, further comprising:
withdrawing the inner catheter; and
returning the medical device to the normal configuration.

26. The method of claim 25, further comprising:
repositioning the distal end the medical device into the patient's aortic arch; and
applying force to a third pull wire to reconfigure the medical device from the normal configuration into the deflected configuration and thereby steer the distal end of the medical device into the patient's proximal left common carotid artery.

27. The method of claim 26, further comprising:
advancing the inner catheter through the medical device and into the patient's left internal carotid artery or the patient's left external carotid artery.

28. The method of claim 27, further comprising:
withdrawing the inner catheter; and
returning the medical device to the normal configuration.

29. The method of claim 28, further comprising:
repositioning the distal end of the medical device into the patient's aortic arch; and
applying force to a fourth pull wire to reconfigure the medical device from the normal configuration into the deflected configuration and thereby steer the medical device into the patient's proximal left subclavian artery.

30. The method of claim 29, further comprising:
advancing the inner catheter through the medical device and into the patient's left vertebral artery.

31. The method of claim 23, wherein applying force to the first pull wire includes deflecting a first steerable segment.

32. The method of claim 21, wherein applying force to the second pull wire includes deflecting a second steerable segment such that the first steerable segment and the second steerable segment are deflected in similar directions.

33. The method of claim 21, wherein applying force to the second pull wire includes deflecting a second steerable segment such that the first steerable segment and the second steerable segment are deflected in generally opposite directions.

34. The method of claim 21, further comprising:
applying force to the at least one pull wire to reconfigure the medical device from the normal configuration into the deflected configuration.

35. The method of claim 34, wherein applying force to the at least one pull wire includes:
applying force to a first pull wire and/or a second pull wire connected to a first steerable segment such that the first steerable segment is deflectable in opposite directions; and
applying force to a third pull wire and/or a fourth pull wire connected to a second steerable segment such that the second steerable segment is deflectable in opposite directions.

36. The method of claim 21, wherein a wheel applies an axial force to the at least one pull wire.

37. A method of establishing trans-radial access to a vascular target site in a patient, the method comprising:
introducing a medical device via a percutaneous opening, wherein the medical device includes:
an outer wall;
a plurality of steerable segments; and
a plurality of pull wires connected to the plurality of steerable segments and extending substantially within the outer wall;
advancing a distal end of the medical device such that the distal end of the medical device is positioned adjacent to an orifice of a target vessel;
reconfiguring the medical device from a normal configuration, in which the medical device is generally linear in configuration, into a deflected configuration, in which the medical device is non-linear in configuration, by applying force to one or more of the plurality of pull wires to thereby steer the medical device towards the vascular target site; and
advancing an inner catheter through the medical device to deliver the inner catheter to the vascular target site.

38. The method of claim 37, wherein introducing the medical device includes introducing the medical device over a rail wire such that the wire extends through medical device.

39. The method of claim 16, wherein advancing the inner catheter includes advancing the inner catheter over the rail wire.

40. The method of claim 37, wherein reconfiguring the medical device includes deflecting the plurality of steerable segments in similar directions.

41. The method of claim 37, wherein reconfiguring the medical device includes deflecting the plurality of steerable segments in dissimilar directions.

42. The method of claim 37, wherein reconfiguring the medical device includes deflecting the plurality of steerable segments in a single direction.

43. The method of claim 37, wherein reconfiguring the medical device includes deflecting the plurality of steerable segments in multiple directions.

44. The method of claim 43, wherein reconfiguring the medical device includes:
deflecting a first steerable segment in a first direction; and
deflecting a second steerable segment in a second direction generally opposite to the first direction.

45. The method of claim 37, wherein reconfiguring the medical device includes:
sequentially applying force to a first pull wire and/or a second pull wire connected to a first steerable segment such that the first steerable segment is deflectable in different directions; and
applying force to a third pull wire and/or a fourth pull wire connected to a second steerable segment such that the second steerable segment is deflectable in different directions.

46. The method of claim 37, wherein reconfiguring the medical device includes:
deflecting a first steerable segment; and
sequentially deflecting a second steerable segment spaced axially from the first steerable segment.

47. The method of claim 37, wherein reconfiguring the medical device includes:
deflecting a first steerable segment; and
sequentially deflecting a second steerable segment, wherein the first steerable segment and the second steerable segment at least partially overlap along a length of the medical device.

48. The method of claim 37, wherein a wheel applies an axial force to the pull wires.

49. A method of establishing trans-radial access to a vascular target site in a patient, the method comprising:
introducing a medical device via a percutaneous opening, wherein the medical device includes:
a body defining a working lumen and including at least one steerable segment; and
at least one pull wire connected to the at least one steerable segment and located substantially within an outer wall of the medical device such that the at least one pull wire is embedded within the medical device;
applying force to the at least one pull wire to reconfigure the medical device and thereby steer the medical device towards the vascular target site; and
subsequent to reconfiguration of the medical device, advancing an inner catheter through the medical device to deliver the inner catheter to the vascular target site.

50. The method of claim 49, wherein advancing the inner catheter includes advancing the inner catheter over a rail wire extending through the medical device.

51. The method of claim 49, wherein applying force to the at least one pull wire includes:
applying force to a first pull wire connected to a first steerable segment; or
applying force to a second pull wire connected to a second steerable segment.

52. The method of claim 49, wherein applying force to the at least one pull wire includes:
applying force to a first pull wire connected to a first steerable segment; and
sequentially applying force to a second pull wire connected to a second steerable segment.

53. The method of claim 49, wherein applying force to the first pull wire and sequentially applying force to the second pull wire includes retracting the first pull wire and retracting the second pull wire within a common channel extending substantially through the outer wall of the medical device in generally parallel relation to the working lumen.

54. The method of claim 49, wherein applying force to the first pull wire includes retracting the first pull wire within a first channel extending substantially through the outer wall of the medical device in generally parallel relation to the working lumen, and sequentially applying force to the second pull wire includes retracting the second pull wire substantially within a second channel extending through the outer wall of the medical device in generally parallel relation to the working lumen.

55. The method of claim 49, wherein applying force to the first pull wire and sequentially applying force to the second pull wire includes deflecting the first steerable segment and deflecting the second steerable segment in generally different directions.

56. The method of claim 49, wherein applying force to the first pull wire and sequentially applying force to the second pull wire includes deflecting the first steerable segment and deflecting the second steerable segment in similar directions.

57. The method of claim 56, wherein applying force to the at least one pull wire includes:
applying force to a first pull wire and/or a second pull wire connected to a first steerable segment such that the first steerable segment is deflectable in different directions; and
applying force to a third pull wire and/or a fourth pull wire connected to a second steerable segment such that the second steerable segment is deflectable in different directions.
